(19) United States
(12) Patent Application Publication
(10) Pub. No.: US 2025/0000778 A1
(43) Pub. Date: Jan. 2, 2025

(54) CHELANT AND CYCLODEXTRIN COMPOSITIONS

(71) Applicant: Coty Inc., New York, NY (US)

(72) Inventors: Claudie Bellouard, Versoix (CH); Jose Maria Velazquez Mendoza, Nyon (CH)

(21) Appl. No.: 18/700,645

(22) PCT Filed: Oct. 11, 2022
(86) PCT No.: PCT/US2022/046284

§ 371 Date: Apr. 11, 2024

Related U.S. Application Data
(60) Provisional application No. 63/254,851, filed on Oct. 12, 2021.

(51) Int. Cl. A61K 8/73; A61Q 13/00

(52) U.S. Cl. CPC A61K 8/738; A61Q 13/00; A61K 2800/51; A61K 2800/592

(57) ABSTRACT

According to various aspects a composition includes a cyclic oligosaccharide; a solvent; a fragrance component; and a chelant. A log K, ion metal constant of the chelant is greater than or equal to 9.

### CROSS-REFERENCE TO RELATED APPLICATIONS

This application claims the benefit of priority to U.S. Provisional Patent Application Ser. No. 63/254,851 entitled “CHELANT AND CYCLODEXTRIN COMPOSITIONS,” filed Oct. 12, 2021, the disclosure of which is incorporated herein in its entirety by reference.

### BACKGROUND

Compositions can include chelants to prevent contaminants from participating in undesirable side reactions. However certain chelants are not compatible with all the components of all compositions and may not dissolve properly therein.

### SUMMARY OF THE DISCLOSURE

According to various aspects a composition includes a cyclic oligosaccharide; a solvent; a fragrance component; and a chelant. A log K, ion metal constant of the chelant is greater than or equal to 9.

According to various aspects a composition includes a cyclic oligosaccharide; a solvent; a fragrance component; and a chelant. The chelant can include GLDA, EDTA, EDDS, MGDA, or a mixture thereof.

According to various aspects a kit includes a container. The container includes a composition, which includes a cyclic oligosaccharide; a solvent; a fragrance component; and a chelant. A log K, ion metal constant of the chelant is greater than or equal to 9.

### BRIEF DESCRIPTION OF THE FIGURES

The drawings illustrate generally, by way of example, but not by way of limitation, various embodiments discussed in the present document.

FIG. 1 is graph showing the correlation between log K of a chelant and dEcmc, in accordance with various embodiments.

FIG. 2 is graph showing the correlation between log K of another chelant and dEcmc, in accordance with various embodiments.

FIG. 3 is graph showing the correlation between log K of another chelant and dEcmc, in accordance with various embodiments.

FIG. 4 is graph showing the correlation between log K of another chelant and dEcmc, in accordance with various embodiments

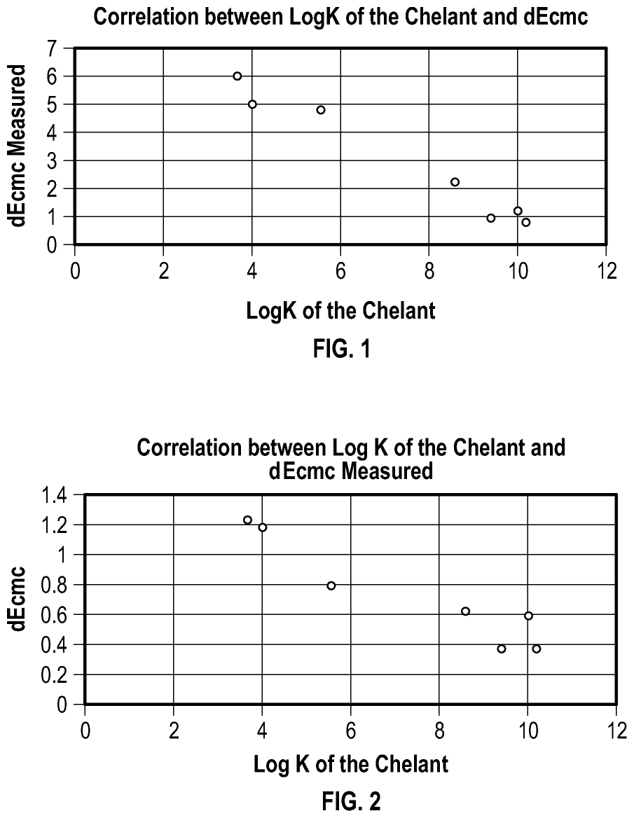

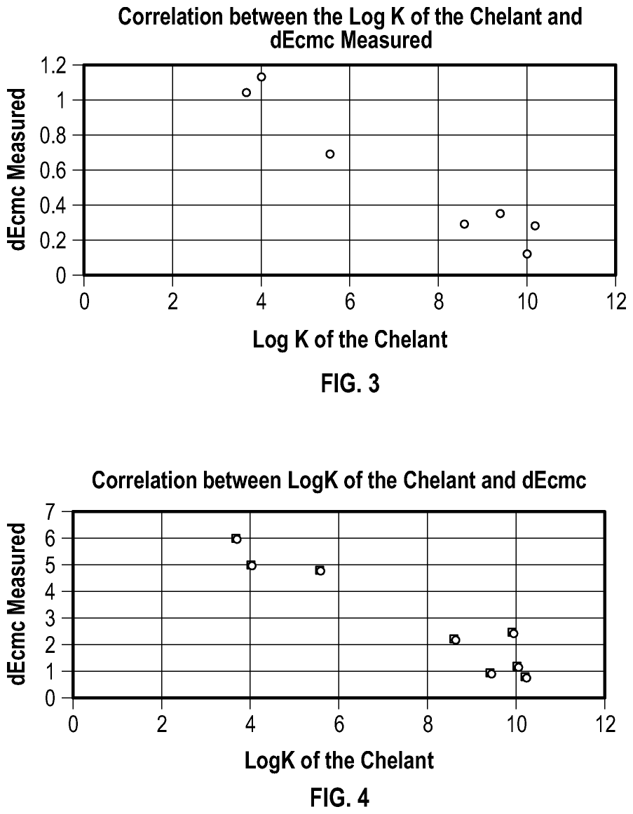

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

The purpose of the disclosed subject matter is to improve stability over time of cosmetic formulations containing volatile solvent with a ratio ethanol/water greater than 7:1, a fragrance and a cyclic oligosaccharide and a chelant having physical properties to allow essentially no interaction with the latter and having a great solubility in the cited medium. The term “improve stability over time” means mainly color stability of samples that were put under accelerated conditions and where the color is checked visually. The term “improve stability over time” also means olfactive stability of the compositions wherein the chelants of the disclosure are used.

Typical cosmetic formulations may include a fragrance and a cyclic oligosaccharide and typically contain lactic acid as chelant. The typical function of a chelant is to remove iron and other contaminants before discoloring reactions occur with sensitive fragrance chemicals. However, in typical constructions using a “common” chelant such as EDTA is not recommended because the solubility in the medium is typically not good enough. However, a lactic acid chelant is shown to provide adequate solubility. However, using lactic acid is not optimal. This is because lactic acid creates some discoloration over time and color change can be undesirable for consumers.

An advancement of this disclosure, relative to the drawbacks noted above, is that cyclic oligosaccharides can be paired with chelants other than lactic acid in cosmetic compositions. This mitigates the discoloration drawbacks associated with using lactic acid. According to various examples a composition can include various components such as a cyclic oligosaccharide; a solvent; a fragrance component; and a chelant. According to various examples, a log K, ion metal constant of the chelant is greater than or equal to 9. Log K is provided by the Public NIST46 Standard Reference—NIST Critically selected stability constants of Metal Complexes database. Examples of compositions that can include these components include a fragrance composition, an antiperspirant, a deodorant, a shampoo, a shower gel, a skin care product, an after shave product, a water-based fragrance composition, or a mixture thereof.

The cyclic oligosaccharide can be present in a range of from about 0.1 wt % to about 50 wt % of the composition, about 0.5 wt % to about 25 wt %, less than, equal to, or greater than about 0.1 wt %, 5, 10, 15, 20, 25, 30, 35, 40, 45, or about 50 wt %.

As used herein, the term “cyclic oligosaccharide” means a cyclic structure comprising six or more saccharide units. Preferred for use herein are cyclic oligosaccharides having six, seven or eight saccharide units and mixtures thereof, more preferably seven saccharide units.

The cyclic oligosaccharides of the present disclosure may comprise any suitable saccharide or mixtures of saccharides. Examples of suitable saccharides include, but are not limited to, glucose, fructose, mannose, galactose, maltose and mixtures thereof. However, preferred for use herein are cyclic oligosaccharides of glucose.

The cyclic oligosaccharides for use herein must have one or more unsubstituted alkyl substituents. The alkyl substituent may be saturated or unsaturated, straight or branched chain but is preferably saturated and straight chain. Preferably, the alkyl substituent is selected from C1-C8 alkyl groups and mixtures thereof, more preferably the alkyl substituent is selected from C1-C6 alkyl groups and mixtures thereof, even more preferably the alkyl substituent is selected from C1-C4 alkyl groups and mixtures thereof. Preferred alkyl substituents are ethyl and methyl, especially methyl.

The cyclic oligosaccharides of the present disclosure are preferably substituted only by the unsubstituted alkyl substituents mentioned hereinabove. However, they may be substituted by other substituents. Examples of other suitable substituents include, but are not limited to, hydroxyalkyl groups, aryl groups, maltosyl groups, allyl groups, benzyl groups, alkanoyl groups and mixtures thereof.

The cyclic oligosaccharides of the present disclosure preferably have an average degree of substitution of from 0.5 to 3.0, more preferably from 1.0 to 2.8, even more preferably from 1.2 to 2.3, especially from 1.6 to 1.9. As used herein the term “degree of substitution” means the average number of substituents per saccharide unit. The average number of substituents can be determined using Nuclear Magnetic Resonance techniques common in the art.

The cyclic oligosaccharides of the present disclosure are preferably soluble in both water and ethanol. As used herein “soluble” means at least 0.1 g of solute dissolves in 100 ml of solvent, at 25° C. and 1 atm of pressure. Preferably the cyclic oligosaccharides for use herein have a solubility of at least 1 g/100 ml, more preferably at least 10 g/100 ml, even more preferably at least 100 g/100 ml, at 25° C. and 1 atm of pressure.

In some examples, the cyclic oligosaccharide can include a cyclodextrin. Examples of suitable cyclodextrins include an α-cyclodextrin, β-cyclodextrin, methyl-α-cyclodextrin, methyl-β-cyclodextrin, or a mixture thereof. In some specific examples the cyclodextrin is methyl-β-cyclodextrin.

The solvent can be present in the composition in any suitable amount. For example, the solvent can be present in a range of from about 1 wt % to about 99 wt % of the composition, about 20 wt % to about 95 wt %, less than, equal to, or greater than about 1 wt %, 5, 10, 15, 20, 25, 30, 35, 40, 45, 50, 55, 60, 65, 70, 75, 80, 85, 90, 95, or about 99 wt % of the composition. Examples of solvents can include an organic solvent, an aqueous solvent, or a mixture thereof. In some examples, where the solvent is an organic solvent, the solvent can be a high volatility organic solvent. In some further examples, the organic solvent can include ethanol. The solvent can also include a mixture of water and ethanol. Other solvents can include Vegetable oils, emollient, glycol and glycol derivatives, glycerol and its derivatives. biology-based hydrocarbons, MIP (esters), non-ionic and ionic surfactants, propanol, n-butyl alcohol, benzyl alcohol, butylene glycol, dipropylene glycol (DPG), diethyl phthalate (DEP), propylene glycol (PG), isopropyl myristate (IPM), isopropyl palmitate (IPP), triethyl citrate (TEC), triacetin (TRI), 1,2-propanediol, 1,3-propanediol, propanethiol, pentanediol, hexanediol, octanediol, decanediol dodecanol, 4-hydroxyacetophenone (glycerol, butylene glycol, pentylene glycol, hexylene glycol, decylene glycol, propylene carbonate, butylene carbonate, glycerol carbonate, 2-benzylheptanol, lauryl alcohol, trimethyl hydroxypentyl isobutyrate, glyceryl caprylate, ethylhexylglycerin, benzyl benzoate (BB), and mixtures thereof.

With respect to the high volatility organic solvent, any volatile solvent suitable for use in the compositions can be used herein. As used herein, “volatile” refers to substances with a significant amount of vapor pressure under ambient conditions, as is understood by those in the art. The volatile solvents for use herein will preferably have a vapor pressure of about 2 kPa or more, more preferably about 6 kPa or more at 25° C. The volatile solvents for use herein will preferably have a boiling point under 1 atm, of less than about 150° C., more preferably less than about 100° C., even more preferably less than about 90° C., even more preferably still less than about 40 80° C. Preferably the volatile solvents for use herein will be safe for use on a wide range of substrates, more preferably on human or animal skin or hair. Suitable volatile solvents include, but are not limited to, those found in the CTFA 45 International Cosmetic Ingredient Dictionary and Handbook, 7th edition, volume 2 P1670-1672, edited by Wenninger and McEwen (The Cosmetic, Toiletry, and Fragrance Association, Inc., Washington, D. C, 1997). Conventionally used volatile solvents include C3-C14 saturated and unsaturated, 50 straight or branched chain hydrocarbons such as cyclohexane, hexane, heptane, isooctane, isopentane, pentane, toluene, xylene; halogenated alkanes such as perfluorodecalin; ethers such as dimethyl ether, diethyl ether; straight or branched chain alcohols and diols such as methanol, ethanol, 55 propanol, isopropanol, n-butyl alcohol, t-butyl alcohol, benzyl alcohol, butoxypropanol, butylene glycol, isopentyldiol; aldehydes and ketones such as acetone; volatile silicones such as cyclomethicones for example octamethyl cyclo tetrasiloxane and decamethyl cyclopentane siloxane; volatile siloxanes 60 such as phenyl pentamethyl disiloxane, phenylethylpentamethyl disiloxane, hexamethyl disiloxane, methoxypropylheptamethyl cyclotetrasiloxane, chloropropyl pentamethyl disiloxane, hydroxypropyl pentamethyl disiloxane, octamethyl cyclotetrasiloxane, decamethyl cyclopentasiloxane; propel-65 lants, and mixtures thereof. Preferred volatile solvents are C1-C4 alcohols and mixtures thereof. More preferred for use herein are C1-C4 straight chain or branched chain alcohols for U.S. Pat. No. 7,820,615 B2 5 example methanol, ethanol, propanol, isopropanol and butanol and mixtures thereof, and most preferred for use herein is ethanol.

When the composition includes a fragrance component, the fragrance component can be in a range of from about 0.01 wt % to about 40 wt % of the composition, about 0.5 wt % to about 25 wt %, less than, equal to, or greater than about 0.01 wt %, 1, 5, 10, 15, 20, 25, 30, 35, or about 40 wt % of the composition. There are many suitable examples of fragrance components that can be included in the composition. In various examples, the fragrance component can include musk oil, civet, castoreum, ambergris, plant fragrances such as nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitrolnellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-β-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox and mixtures thereof. The chelant can be present in the composition in any suitable amount. For example the chelant can be present in an amount of from about 20 wt part per one wt part of metal present to about 80 wt part per one wt part of metal present of the composition, about 40 wt part per one wt part of metal present to about 70 wt part per one wt part of metal present, less than, equal to, or greater than about 20 wt part per one wt part of metal present, 30, 40, 50, 60, 70, or about 80 wt part per one wt part of metal present. In some examples, the composition can include a mixture of two or more chelants. Examples of suitable chelants can include tetrasodium glutamate diacetate (GLDA), ethylenediaminetetraacetic acid (EDTA), ethylenediamine-N, N′-disuccinic acid (EDDS), NTA, EDDS, IDS, methylglycinediacetic acid (MGDA), L-glutamic acid N,N-diacetic acid (GLDA), ethylenediamine-N,N-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), methylglycine N,N-diacetic acid trisodium salt (MGDA), or a mixture thereof.

The selection of a specific chelant can be dictated by its log K, ion metal constant (alternatively simply referred to as a “log K”). For example, according to various examples, the log K, ion metal constant of the chelant is greater than 9, greater than 10, or greater than 15. It has been found that if the chelant has the above indicated log K, ion metal constant, functions well in the presence of a cyclic oligosaccharide to 1) dissolve in solution; and 2) prevent the above indicated undesirable discoloration in the composition.

The compositions herein can contain a variety of other optional components suitable for rendering such compositions more cosmetically or aesthetically acceptable or to provide them with additional usage benefits. Such conventional optional ingredients are well-known to those skilled in the art. These include any cosmetically acceptable ingredients such as those found in the CTFA International Cosmetic Ingredient Dictionary and Handbook, 7th edition, edited by Wenninger and McEwen, (The Cosmetic, Toiletry, and Fragrance Association, Inc., Washington, D.C., 1997). As used herein “cosmetically acceptable” means a material (e.g., compound or composition) which is suitable for use in contact with skin, hair or other suitable substrate as defined hereinbelow.

### EXAMPLES

Various embodiments of the present disclosure can be better understood by reference to the following Examples which are offered by way of illustration. The present disclosure is not limited to the Examples given herein.

### Example 1

Composition A
Chelant of the disclosure with a log K equal to 9.4

| Ingredient | % weight |
|---|---|
| Water | 4.5-7.5 |
| GLDA | 0.0001-0.002 |
| Ethanol | 70.0-80.05 |
| Perfume A (1) | 5-9 |
| Cyclodextrin in solution (2) | 10-14 |
| Dyes (blue) | 0.30-0.70 |

(1) Perfume commercialized and supplied by Firmenich
(2) Cawasol supplied by Wacker

Composition B
Chelant of the disclosure with a log K equal to 10.01

| Ingredient | % weight |
|---|---|
| Water | 4.5-7.5 |
| EDDS | 0.001-0.005 |
| Ethanol | 70.0-80.0 |
| Perfume A (1) | 5-9 |
| Cyclodextrin in solution (2) | 10-14 |
| Dyes (blue) | 0.30-0.70 |

(1) Perfume commercialized and supplied by Firmenich
(2) Cawasol supplied by Wacker

Composition C
Chelant of the disclosure with a log K equal to 10.19

| Ingredient | % weight |
|---|---|
| Water | 4.5-6.5 |
| EDTA | 0.0003-0.0009 |
| Ethanol | 70.0-80.0 |
| Perfume A (1) | 5-9 |
| Cyclodextrin in solution (2) | 10.0-14.0 |
| Dyes (blue) | 0.35-0.75 |

(1) Perfume commercialized and supplied by Firmenich
(2) Cawasol supplied by Wacker

Comparative Composition D
Chelant with a log K equal to 3.67

| Ingredient | % weight |
|---|---|
| Water | 3.0-6.0 |
| Lactic acid | 0.07-0.10 |
| NaOH 32% solution in water | 0.05-0.09 |
| Ethanol | 72.0-77.0 |
| Perfume A (1) | 5-9 |
| Cyclodextrin in solution (2) | 10.0-14.0 |
| Dyes (blue) | 0.30-0.70 |

(1) Perfume commercialized and supplied by Firmenich
(2) Cawasol supplied by Wacker

### Color Stability

Color stability is assessed by experts who rate the color stability of sample stored for 8 weeks at 45° C. versus the reference stored for 8 weeks at 5° C. They use a scale from A to C wherein A means the sample shows same color as the reference, B means the sample shows slight color change over the reference and passes the stability test and C, means the sample shows great color change over the reference and the sample fails the stability test.

The dEcmc is also measured on each sample of the different compositions after 8 weeks storage at 45° C. Greater the dEcmc is, higher the instability of the sample is. The results are summarized in the table below.

| | Com- | Com- | Com- | |
|---|---|---|---|---|
| | position A | position B | position C | |
| | with the | with the | with the | |
| | chelant | chelant | chelant | |
| After 8 weeks | of the | of the | of the | Com- |
| storage | disclosure | disclosure | disclosure | position D |
| Expert evaluation | B | B | B | C |
| dEcmc calculated | 1.19 | 5.14 | 6.81 | 17.94 |
| versus the | | | | |
| reference | | | | |

The 3 compositions of the disclosure A, B and C show an improved color stability in the presence of cyclodextrin while the composition D having lactic acid as a chelant shows a great color change versus the reference thus is considered as not stable by experts.

### Olfactive Stability:

Stability of the fragrance is assessed by experts who evaluate the intensity and the character of the tested fragrance stored for 8 weeks at 45° C. versus the reference stored for 8 weeks at 5° C. Then they rate the stability on a scale from 1 to 5 wherein scores of 1, 2 and 3 mean the fragrance shows an important change in intensity and character over the reference, a score of 4 means the fragrance shows slight change in intensity or character over the reference and a score of 5, means the fragrance shows no change in intensity and character over the reference. The results are summarized in the table below

| | Com- | Com- | Com- | |
|---|---|---|---|---|
| | position A | position B | position C | |
| After 8 weeks | of the | of the | of the | Com- |
| storage | disclosure | disclosure | disclosure | position D |
| Expert evaluation | 4 | 4 | 4 | 3 |

The compositions of the disclosure A, B and C show an improved olfactive stability in the presence of cyclodextrin while the composition having lactic acid as a chelant shows a great change in the fragrance profile thus is considered as not stable by experts.

### Example 2

This example examines GLDA (composition A) versus other comparative chelants (lactic acid, citric acid, and benzoate) that are not claimed—Crash Test 1 week storage at 55° C.

Composition A
Chelant of the disclosure with a log K equal to 9.4

| Ingredient | % weight |
|---|---|
| Water | 5.3-5.9 |
| Chelant: GLDA | 0.0005-0.003 |
| Ethanol | 70.0-77.0 |
| Perfume A (1) | 5-9 |
| Cyclodextrin in solution (2) | 10.0-14.0 |
| Dyes (blue) | 0.30-0.70 |

(1) Perfume commercialized and supplied by Firmenich
(2) Cawasol supplied by Wacker

Composition D
Chelant with a log K equal to 3.67

| Ingredient | % weight |
|---|---|
| Water | 2.0-7.0 |
| Lactic acid | 0.07-0.85 |
| NaOH 32% solution in water | 0.05-0.09 |
| Ethanol | 65.0-80.0 |
| Perfume A (1) | 5-9 |
| Cyclodextrin in solution (2) | 10.0-13.0 |
| Dyes (blue) | 0.35-0.70 |

(1) Perfume commercialized and supplied by Firmenich
(2) Cawasol supplied by Wacker

Composition E
Chelant with a log K equal to 5.56

| Ingredient | % weight |
|---|---|
| Water | 3.0-6.0 |
| Chelant: Citric acid | 0.005-0.05 |
| Ethanol | 70.0-80.0 |
| Perfume A (1) | 5-9 |
| Cyclodextrin in solution (2) | 10.0-15.0 |
| Dyes (blue) | 0.30-0.70 |

(1) Perfume commercialized and supplied by Firmenich
(2) Cawasol supplied by Wacker

Composition F
Chelant with a log K equal to

| Ingredient | % weight |
|---|---|
| Water | 3.0-7.0 |
| Chelant: Sodium benzoate | 0.005-0.03 |
| Ethanol | 70.0-80.0 |
| Perfume A | 5-9 |
| Cyclodextrin in solution* | 10.0-15.0 |
| Dyes (blue) | 0.35-0.80 |

(1) Perfume commercialized and supplied by Firmenich
(2) Cawasol supplied by Wacker

### Color Stability

A crash test was conducted with a storage at 55° C. for 1 week. Then the samples were rated by experts.

Color stability is assessed by experts who rate the color stability of sample stored at high temperature versus the reference at 5° C. They use a scale from A to C wherein A means the sample shows same color as the reference, B means the sample shows slight color change over the reference and passes the stability test and C, means the sample shows great color change over the reference and the sample fails the stability test.

The dEcmc is also measured on each sample of the different compositions in the same conditions as for the crash test, so after 1 week storage at 55° C. Greater the dEcmc is, higher the instability of the sample is.

The results are summarized in the table below.

| | Com- | | | |
|---|---|---|---|---|
| | position A | | | |
| | Chelant | | | |
| | of the | Com- | Com- | Com- |
| Crash Test | disclosure | position D | position E | position F |
| Expert evaluation | B | C | C | C |
| dEcmc calculated | 0.95 | 6 | 4.8 | 5 |
| versus the | | | | |
| reference | | | | |

The composition of the disclosure A shows an improved color stability while the compositions D,E and F that do not use the chelants of the disclosure shows a high instability thus are considered as not stable by experts.

Log K: This demonstrates the competing mechanism between the metal ion and the cyclodextrin. Chelants with a weak association to the metal ion (log K<9.4) will reach an equilibrium between the CD and the Ion. Therefore, the colour will change (potentially reversibly depending on light or temperature that change the enthalpy of the system). The equation for the correlation coefficient is:

$$Correl ( X , Y ) = \frac{\sum ( x - \overline{x} ) \sum ( y - \overline{y} )}{\sqrt{\sum {( x - \overline{x} )}^{2} \sum {( y - \overline{y} )}^{2}}}$$

Where x̅ and y̅ are the sample means Average (array 1) and AVERAGE (array 2)

The table below summarizes the dEcmc of a composition using perfume A:

| | | dEcmc measured |
|---|---|---|
| | Log K | after 1 week at 55° C. |
| lactic acid | 3.67 | 6 |
| Benzoic Acid | 4.01 | 5 |
| citric acid | 5.56 | 4.8 |
| Dermofeel 12 | 8.59 | 2.23 |
| GLDA (organic | 9.4 | 0.95 |
| derivative) | | |
| EDDS | 10.01 | 1.2 |
| EDTA | 10.19 | 0.8 |
| Correlation | −0.98 | |

### Example 3: Testing with Different Perfumes and Different Colors

### Perfume B

Composition G

| Ingredient | % weight |
|---|---|
| Water | 6.0-9.0 |
| Chelant: GLDA | 0.0005-0.005 |
| Ethanol | 70.0-85.0 |
| Perfume B (1) | 3.5-5.5 |
| Cyclodextrin in solution (2) | 10.5-15.0 |
| Dyes (Intense pink) | 0.10-0.30 |

(1) Perfume commercialized and supplied by Firmenich
(2) Cawasol supplied by Wacker

Composition H

| Ingredient | % weight |
|---|---|
| Water | 5.0-9.0 |
| Chelant: EDDS | 0.001-0.005 |
| Ethanol | 70.0-79.0 |
| Perfume B (1) | 3.5-6.5 |
| Cyclodextrin in solution (2) | 10.0-14.0 |
| Dyes (Intense pink) | 0.15-0.35 |

(1) Perfume commercialized and supplied by Firmenich
(2) Cawasol supplied by Wacker

Composition I

| Ingredient | % weight |
|---|---|
| Water | 5.0-9.0 |
| Chelant: EDTA | 0.0002-0.0009 |
| Ethanol | 70.0-80.0 |
| Perfume B (1) | 3-7 |
| Cyclodextrin in solution (2) | 10.0-15.0 |
| Dyes (Intense pink) | 0.10-0.40 |

(1) Perfume commercialized and supplied by Firmenich
(2) Cawasol supplied by Wacker

Composition J

| Ingredient | % weight |
|---|---|
| Water | 5.0-8.0 |
| Chelant: Lactic acid | 0.05-0.2 |
| NaOH 32% solution in water | 0.03-0.09 |
| Ethanol | 70.0-80.0 |
| Perfume B (1) | 3-7 |
| Cyclodextrin in solution (2) | 10.0-15.0 |
| Dyes (Intense pink) | 0.15-0.30 |

(1) Perfume commercialized and supplied by Firmenich
(2) Cawasol supplied by Wacker

### Color Stability

Color stability is assessed by experts who rate the color stability of sample stored for 8 weeks at 45° C. versus the reference stored for 8 weeks at 5° C. They use a scale from A to C wherein A means the sample shows same color as the reference, B means the sample shows slight color change over the reference and passes the stability test and C, means the sample shows great color change over the reference and the sample fails the stability test.

The dEcmc is also measured on each sample of the different compositions after 8 weeks storage at 45° C. Greater the dEcmc is, higher the instability of the sample is. The results are summarized in the table below.

| | Com- | Com- | Com- | |
|---|---|---|---|---|
| | position G | position H | position I | |
| | Chelant | Chelant | Chelant | |
| After 8 weeks | of the | of the | of the | Com- |
| storage | disclosure | disclosure | disclosure | position J |
| Expert evaluation | B | B | B | C |
| dEcmc calculated | 2.55 | 1.68 | 1.14 | 3.24 |
| versus the | | | | |
| reference | | | | |

The three compositions of the disclosure show an improved stability in the presence of cyclodextrin while the composition having lactic acid as a chelant shows the highest score in color change versus the reference thus is considered as not stable by experts.

Perfume B (color: Pink intense)

| | | dEcmc measured |
|---|---|---|
| | | after 1 week at |
| | Log K | 55° C. |
| lactic acid | 3.67 | 1.23 |
| Benzoic Acidx | 4.01 | 1.18 |
| citric acid | 5.56 | 0.79 |
| Dermofeel 12 | 8.59 | 0.62 |
| GLDA | 9.4 | 0.37 |
| EDDS | 10.01 | 0.59 |
| EDTA | 10.19 | 0.37 |

| | |
|---|---|
| correlation | −0.94761 |

### Perfume C

Composition K

| Ingredient | % weight |
|---|---|
| Water | 5.0-9.0 |
| Chelant: GLDA | 0.0005-0.005 |
| Ethanol | 70.0-80.0 |
| Perfume C (1) | 3-7 |
| Cyclodextrin in solution (2) | 10.0-15.0 |
| Dyes (light grey) | 0.20-0.29 |

(1) Perfume commercialized and supplied by Givaudan
(2) Cawasol supplied by Wacker

Composition L

| Ingredient | % weight |
|---|---|
| Water | 6.5-9.0 |
| Chelant: EDDS | 0.001-0.006 |
| Ethanol | 70.0-80.0 |
| Perfume C (1) | 3-7 |
| Cyclodextrin in solution (2) | 9.0-15.0 |
| Dyes (light grey) | 0.10-0.30 |

(1) Perfume commercialized and supplied by Givaudan
(2) Cawasol supplied by Wacker

Composition M

| Ingredient | % weight |
|---|---|
| Water | 6.5-9.5 |
| EDTA | 0.0001-0.003 |
| Ethanol | 70.0-80.0 |
| Perfume C (1) | 3-7 |
| Cyclodextrin in solution (2) | 10.0-15.0 |
| Dyes (light grey) | 0.15-0.30 |

(1) Perfume commercialized and supplied by Givaudan
(2) Cawasol supplied by Wacker

Comparative Composition N

| Ingredient | % weight |
|---|---|
| Water | 5.9-9.0 |
| Lactic acid | 0.05-0.20 |
| NaOH 32% solution in water | 0.05-0.09 |
| Ethanol | 70.0-80.0 |
| Perfume C (1) | 3-7 |
| Cyclodextrin in solution (2) | 10-15 |
| Dyes (light grey) | 0.1-0.4 |

(1) Perfume commercialized and supplied by Givaudan
(2) Cawasol supplied by Wacker

Comparative Composition O

| Ingredient | % weight |
|---|---|
| Water | 5.0-9.0 |
| Chelant: Citric acid | 0.05-0.20 |
| NaOH 32% solution in water | 0.05-0.20 |
| Ethanol | 70.0-80.0 |
| Perfume C (1) | 3-7 |
| Cyclodextrin in solution (2) | 10.0-15.0 |
| Dyes (light grey) | 0.05-0.30 |

(1) Perfume commercialized and supplied by Givaudan
(2) Cawasol supplied by Wacker

### Color Stability

Color stability is assessed by the measurement of the dEcmc on each sample of the different compositions in the same conditions as for the crash test, so after 1 week storage at 55° C. Greater the dEcmc is, higher the instability of the sample is. The results are summarized in the table below.

| | Com- | Com- | Com- | | |
|---|---|---|---|---|---|
| | position K | position L | position M | | |
| | Chelant | Chelant | Chelant | | |
| | of the | of the | of the | Com- | Com- |
| Crash test | disclosure | disclosure | disclosure | position N | position O |
| dEcmc | 0.35 | 0.12 | 0.28 | 1.04 | 0.69 |
| calculated | | | | | |
| versus the | | | | | |
| reference | | | | | |

The three compositions of the disclosure show an improved stability in the presence of cyclodextrin while the composition comprising the chelants not from the disclosure show the highest score in color change.

| | | dEcmc measured |
|---|---|---|
| | | after 1 week at |
| | Log K | 55° C. |
| lactic acid | 3.67 | 1.04 |
| Benzoic Acid | 4.01 | 1.13 |
| citric acid | 5.56 | 0.69 |
| Dermofeel 12 | 8.59 | 0.29 |
| GLDA | 9.4 | 0.35 |
| EDDS | 10.01 | 0.12 |
| EDTA | 10.19 | 0.28 |

| | |
|---|---|
| Correlation | −0.97 |

Composition P shows that MGDA can be used as a chelant.

Composition P

| Ingredient | % weight |
|---|---|
| Water | 4.5-7.5 |
| MGDA | 0.001-0.05 |
| Ethanol | 70.0-80.0 |
| Perfume A (1) | 5-9 |
| Cyclodextrin in solution (2) | 10.0-14.0 |
| Dyes (blue) | 0.30-0.70 |

(1) Perfume commercialized and supplied by Firmenich
(2) Cavasol supplied by Wacker

The color stability of Composition P is assessed by the measurement of the dEcmc on each sample of the different compositions in the same conditions as for the crash test, so after 1 week storage at 55° C. Greater the dEcmc is, higher the instability of the sample is. The results are summarized in the table below.

| | Composition Y |
|---|---|
| Crash test | Chelant of the disclosure |
| dEcmc calculated versus the | 2.47 |
| reference | |

The table below summarizes the dEcmc of a composition using perfume A:

| | | dEcmc measured |
|---|---|---|
| | | after 1 week at |
| | Log K | 55° C. |
| lactic acid | 3.67 | 6 |
| Benzoic Acid | 4.01 | 5 |
| citric acid | 5.56 | 4.8 |
| Dermofeel 12 | 8.59 | 2.23 |
| GLDA (organic | 9.4 | 0.95 |
| derivative) | | |
| MGDA | 9.9 | 2.47 |
| EDDS | 10.01 | 1.2 |
| EDTA | 10.19 | 0.8 |

| | |
|---|---|
| Correlation | −0.96 |

The use of the chelants described herein allow for a fragrance chassis to be developed that uses lower levels of ethanol (65-70 wt %) as compared to a conventional fragrance chassis. An example of such a chassis is provided below.

Low VOC chassis 1 with the chelant of the invention

| Ingredient | % weight |
|---|---|
| Water | 10-15 |
| GLDA | 0.0005-0.003 |
| Ethanol | 65-70 |
| Perfume | 5-9 |
| Metyl-B-Cyclodextrin in solution (1) | 10.0-14.0 |
| Dyes (blue) | 0.30-0.70 |

(1) Cavasol supplied by Wacker

Expert olfactive assessment after 2 months storage: 5A- for the low VOC chassis with the chelant from the invention. 3B- for the original formula

Additionally, it is shown that the chelants can be incorporated in a water-based (e.g., water solvent) fine fragrance. Examples of water-based fine fragrances are provided below.

Water based FF Composition A:

| Ingredient | % weight |
|---|---|
| PPG-26-Buteth-26 (and) PEG-40 Hydrogenated | 10-20 |
| Castor Oil in solution (1) | |
| Propylene glycol | 2-5 |
| Perfume | 5-9 |
| Deionized water | 60-70 |
| GLDA | 0.0005-0.003 |
| Metyl-B-Cyclodextrin in solution (2) | 10.0-14.0 |
| Dyes (blue) | 0.30-0.70 |

(1) Solubilisant LRI supplied by Sensient
(2) Cavasol supplied by Wacker

Water based FF Composition B:

| Ingredient | % weight |
|---|---|
| PPG-26-Buteth-26 (and) PEG-40 Hydrogenated | 10-20 |
| Castor Oil in solution (1) | |
| Propylene glycol | 2-5 |
| Perfume | 5-9 |
| Deionized water | 60-70 |
| Citric acid | 0.05-0.1 |
| Metyl-B-Cyclodextrin in solution (2) | 10.0-14.0 |
| Dyes (blue) | 0.30-0.70 |

(1) Solubilisant LRI supplied by Sensient
(2) Cavasol supplied by Wacker

Water based FF Composition C:

| Ingredient | % weight |
|---|---|
| PPG-26-Buteth-26 (and) PEG-40 Hydrogenated | 10-20 |
| Castor Oil in solution (1) | |
| Propylene glycol | 2-5 |
| Perfume | 5-9 |
| Deionized water | 60-70 |
| Iminodisuccinic acid | 0.05-0.1 |
| Metyl-B-Cyclodextrin in solution (2) | 10.0-14.0 |
| Dyes (blue) | 0.30-0.70 |

(1) Solubilisant LRI supplied by Sensient
(2) Cavasol supplied by Wacker

Water based FF Composition D

| Ingredient | % weight |
|---|---|
| PPG-26-Buteth-26 (and) PEG-40 Hydrogenated | 10-20 |
| Castor Oil in solution (1) | |
| Propylene glycol | 2-5 |
| Perfume | 5-9 |
| Deionized water | 60-70 |
| MGDA | 0.05-0.1 |
| Metyl-B-Cyclodextrin in solution (2) | 10.0-14.0 |
| Dyes (blue) | 0.30-0.70 |

(1) Solubilisant LRI supplied by Sensient
(2) Cavasol supplied by Wacker

Water based FF Composition E:

| Ingredient | % weight |
|---|---|
| PPG-26-Buteth-26 (and) PEG-40 Hydrogenated | 10-20 |
| Castor Oil in solution (1) | |
| Propylene glycol | 2-5 |
| Perfume | 5-9 |
| Deionized water | 65-75 |
| GLDA | 0.0005-0.003 |
| alpha-Cyclodextrin | 0.1-2.0 |
| Dyes (blue) | 0.30-0.70 |

(1) Solubilisant LRI supplied by Sensient

Color stability is assessed by the measurement of the dEcmc on each sample of the different compositions in the same conditions as for the crash test, so after 1 week storage at 55° C. Greater the dEcmc is, higher the instability of the sample is. The results are summarized in the table below.

| | Water base FF | | Water base FF | Water base FF | Water base FF |
|---|---|---|---|---|---|
| | Composition A: | | Composition C: | Composition D: | Composition E: |
| | Chelant of the | Water base FF | Chelant of the | Chelant of the | Chelant of the |
| Crash test | disclosure | Composition B: | disclosure | disclosure | disclosure |
| dEcmc | 2.39 | 6.46 | 2.69 | 3.00 | 2.43 |
| calculated | | | | | |
| versus the | | | | | |
| reference | | | | | |

The compositions of the disclosure show an improved stability in the presence of cyclodextrin while the composition comprising the chelant not from the disclosure show the highest score in color change. The chelants described herein can also allow for the construction of various antiperspirant roll-on compositions. Examples of suitable antiperspirant roll-on compositions include those provided below:

Roll-on composition A:

| Ingredient | % weight |
|---|---|
| Deionized water | 72.0-78.0 |
| GLDA | 0.0005-0.003 |
| Structure XL | 1.0-5.0 |
| ALUMINUM SALT in solution | 10.0-20.0 |
| AP Moisture complex | 0.001-0.5 |
| Perfume | 0.5-2.0 |
| Metyl-B-Cyclodextrin in solution (1) | 0.5-2.0 |

(1) Cavasol supplied by Wacker

Comparative Roll-on composition B:

| Ingredient | % weight |
|---|---|
| Deionized water | 72.0-78.0 |
| Citric acid | 0.001-0.2 |
| Structure XL | 1.0-5.0 |
| ALUMINUM SALT in solution | 10.0-20.0 |
| AP Moisture complex | 0.001-0.5 |
| Perfume | 0.5-2.0 |
| Metyl-B-Cyclodextrin in solution (1) | 0.5-2.0 |

(1) Cavasol supplied by Wacker

Color stability is assessed by the measurement of the dEcmc on each sample of the different compositions in the same conditions as for the crash test, so after 1 week storage at 55° C. Greater the dEcmc is, higher the instability of the sample is. The results are summarized in the table below.

| | Roll-on composition A | Roll-on |
|---|---|---|
| Crash test | Chelant of the disclosure | composition B |
| dEcmc calculated versus | 3.03 | 3.72 |
| the reference | | |

The composition of the disclosure shows an improved stability in the presence of cyclodextrin while the composition comprising the chelant not from the disclosure show the highest score in color change.

Additionally, the chelants described herein can be used in in conjunction with an after shave composition. Examples of suitable after shave compositions are provided below.

After shave composition A:

| Ingredient | % weight |
|---|---|
| Ethanol | 50-55 |
| GLDA | 0.0005-0.003 |
| PEG-Hydrogenated castor oil | 2-5 |
| Water | 30-35 |
| Allantoin | 0.1-0.2 |
| Perfume | 1-3 |
| Metyl-B-Cyclodextrin in solution (1) | 10-14 |
| Dyes in solution | 0.3-0.7 |

(1) Cavasol supplied by Wacker

Comparative After shave composition B:

| Ingredient | % weight |
|---|---|
| Ethanol | 50-55 |
| Lactic acid | 0.01-0.2 |
| PEG-Hydrogenated castor oil | 2-5 |
| Water | 30-35 |
| Allantoin | 0.1-0.2 |
| Perfume | 1-3 |
| Metyl-B-Cyclodextrin in solution (1) | 10-14 |
| Dyes in solution | 0.3-0.7 |

(1) Cavasol supplied by Wacker

Color stability is assessed by the measurement of the dEcmc on each sample of the different compositions in the same conditions as for the crash test, so after 1 week storage at 55° C. Greater the dEcmc is, higher the instability of the sample is. The results are summarized in the table below.

| | After shave composition A | After shave |
|---|---|---|
| Crash test | Chelant of the disclosure | composition B |
| dEcmc calculated versus | 0.32 | 4.65 |
| the reference | | |

The composition of the disclosure shows an improved stability in the presence of cyclodextrin while the composition comprising the chelant not from the disclosure show the highest score in color change.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Aspects.

The following exemplary aspects are provided, the numbering of which is not to be construed as designating levels of importance:

Aspect 1 provides a composition comprising:
a cyclic oligosaccharide;
a solvent;
a fragrance component;
a chelant, wherein a log K, ion metal constant of the chelant is greater than or equal to 9.

Aspect 2 provides the fragrance composition of Aspect 1, wherein the cyclic oligosaccharide comprises about 0.1 wt % to about 50 wt % of the fragrance composition.

Aspect 3 provides the fragrance composition of any one of Aspects 1 or 2, wherein the cyclic oligosaccharide comprises about 0.5 wt % to about 25 wt % of the fragrance composition.

Aspect 4 provides the fragrance composition of any one of Aspects 1-3, wherein the cyclic oligosaccharide comprises a cyclodextrin.

Aspect 5 provides the fragrance composition of any one of Aspects 1-4, wherein the cyclic oligosaccharide comprises an α-cyclodextrin, β-cyclodextrin, methyl-α-cyclodextrin, methyl-β-cyclodextrin, or a mixture thereof.

Aspect 6 provides the fragrance composition of Aspect 5, wherein the cyclodextrin is methyl-β-cyclodextrin.

Aspect 7 provides the fragrance composition of any one of Aspects 1-6, wherein the solvent comprises about 1 wt % to about 99 wt % of the fragrance composition.

Aspect 8 provides the fragrance composition of any one of Aspects 1-7, wherein the solvent comprises about 20 wt % to about 95 wt % of the fragrance composition.

Aspect 9 provides the fragrance composition of any one of Aspects 1-8, wherein the solvent comprises an organic solvent, an aqueous solvent, or a mixture thereof.

Aspect 10 provides the fragrance composition of Aspect 9, wherein the solvent is an organic solvent and is high volatility organic solvent.

Aspect 11 provides the fragrance composition of any one of Aspects 9 or 10, wherein the organic solvent comprises ethanol.

Aspect 12 provides the fragrance composition of any one of Aspects 9-11, wherein the solvent comprises a mixture of water and ethanol.

Aspect 13 provides the fragrance composition of any one of Aspects 1-12, wherein the fragrance component is in a range of from about 0.01 wt % to about 40 wt % of the fragrance composition.

Aspect 14 provides the fragrance composition of any one of Aspects 1-13, wherein the fragrance component is in a range of from about 0.5 wt % to about 25 wt % of the fragrance composition.

Aspect 15 provides the fragrance composition of any one of Aspects 1-14, wherein the fragrance component comprises musk oil, civet, castoreum, ambergris, plant fragrances such as nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitrolnellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-β-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox and mixtures thereof.

Aspect 16 provides the fragrance composition of any one of Aspects 1-15, wherein the chelant is in a range of from about 20 wt part per one wt part of metal present to about 80 wt part per one wt part of metal present of the fragrance composition.

Aspect 17 provides the fragrance composition of any one of Aspects 1-16, wherein the chelant is in a range of from about 40 wt part per one wt part of metal present to about 70 wt part per one wt part of metal present of the fragrance composition.

Aspect 18 provides the fragrance composition of any one of Aspects 1-17, wherein the chelants comprises a mixture of two or more chelants.

Aspect 19 provides the fragrance composition of any one of Aspects 1-18, wherein the chelant comprises GLDA, EDTA, MGDA, EDDS, or a mixture thereof.

Aspect 20 provides the fragrance composition of any one of Aspects 1-19, wherein the log K, ion metal constant of the chelant is greater than 9.

Aspect 21 provides the fragrance composition of any one of Aspects 1-20, wherein the log K, ion metal constant of the chelant is greater than 10.

Aspect 22 provides the fragrance composition of any one of Aspects 1-21, wherein the log K, ion metal constant of the chelant is greater than 15.

Aspect 23 provides a fragrance composition comprising:
a cyclodextrin;
a solvent comprising water, ethanol, or a mixture thereof;
a fragrance component;
a chelant comprising GLDA, EDTA, MGDA, EDDS, or a mixture thereof.

Aspect 24 provides a kit comprising:
a container; and
the fragrance composition of any one of Aspects 1-23 disposed within the composition.

1. A composition comprising:
a cyclic oligosaccharide in a range of from greater than 0.5 wt % to about 25 wt % of the composition;
a solvent;
a fragrance component;
a chelant comprising GLDA, EDTA, EDDS, MGDA or a mixture thereof, wherein a log K, ion metal constant of the chelant is greater than or equal to 9.

2. (canceled)

3. (canceled)

4. The fragrance composition of claim 1, wherein the cyclic oligosaccharide comprises a cyclodextrin.

5. The fragrance composition of claim 1, wherein the cyclic oligosaccharide comprises an α-cyclodextrin, β-cyclodextrin, methyl-α-cyclodextrin, methyl-β-cyclodextrin, or a mixture thereof.

6. The fragrance composition of claim 5, wherein the cyclodextrin is methyl-β-cyclodextrin.

7. The fragrance composition of claim 1, wherein the solvent comprises about 1 wt % to about 99 wt % of the fragrance composition.

8. The fragrance composition of claim 1, wherein the solvent comprises about 20 wt % to about 95 wt % of the fragrance composition.

9. The fragrance composition of claim 1 wherein the solvent comprises an organic solvent, an aqueous solvent, or a mixture thereof.

10. The fragrance composition of claim 9, wherein the solvent is an organic solvent and is high volatility organic solvent.

11. The fragrance composition of claim 9, wherein the organic solvent comprises ethanol.

12. The fragrance composition of claim 9, wherein the solvent comprises a mixture of water and ethanol.

13. The fragrance composition of claim 1, wherein the fragrance component is in a range of from about 0.01 wt % to about 40 wt % of the fragrance composition.

14. The fragrance composition of claim 1, wherein the fragrance component is in a range of from about 0.5 wt % to about 25 wt % of the fragrance composition.

15. The fragrance composition of claim 1, wherein the fragrance component comprises musk oil, civet, castoreum, ambergris, plant fragrances such as nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitrolnellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-β-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox and mixtures thereof.

16. The fragrance composition of claim 1, wherein the chelant is in a range of from about 20 wt part per one wt part of metal present to about 80 wt part per one wt part of metal present of the fragrance composition.

17. The fragrance composition of claim 1, wherein the chelant is in a range of from about 40 wt part per one wt part of metal present to about 70 wt part per one wt part of metal present of the fragrance composition.

18. The fragrance composition of claim 1, wherein the chelants comprises a mixture of two or more chelants.

19. The fragrance composition of claim 1, wherein the chelant comprises GLDA, EDTA, EDDS, or MGDA.

20. The fragrance composition of claim 1, wherein the log K, ion metal constant of the chelant is greater than 9.

21. The fragrance composition of claim 1, wherein the log K, ion metal constant of the chelant is greater than 10.

22. (canceled)

23. A fragrance composition comprising:
a cyclodextrin;
a solvent comprising water, ethanol, or a mixture thereof;
a fragrance component;
a chelant comprising GLDA, EDTA, EDDS, MGDA, or a mixture thereof.

24. (canceled)
